(19) United States
(12) Patent Application Publication
(10) Pub. No.: US 2013/0041272 A1
(43) Pub. Date: Feb. 14, 2013

(54) SENSOR APPARATUS ADAPTED TO BE INCORPORATED IN A GARMENT

(71) Applicants: Javier Guillén Arredondo, Benaguasil (Valencia) (ES); Sergio Guillén Barrionuevo, Benaguasil (Valencia) (ES)

(72) Inventors: Javier Guillén Arredondo, Benaguasil (Valencia) (ES); Sergio Guillén Barrionuevo, Benaguasil (Valencia) (ES)

(73) Assignee: WEARABLE INFORMATION TECHNOLOGIES, S.L. (WEARTECH), BENAGUASIL (Valencia) (ES)

(21) Appl. No.: 13/642,496

(22) PCT Filed: Apr. 20, 2010
(86) PCT No.: PCT/EP2010/055224

§ 371 Date: Oct. 26, 2012

(51) Int. Cl. A61B 5/0402; B32B 38/00; A61B 5/08; A41D 1/00; A61B 5/00

(52) U.S. Cl. 600/509; 2/69; 600/300; 600/534; 156/93

(57) ABSTRACT

A sensor apparatus adapted to be incorporated in a garment, said apparatus comprising one or more sensors, one or more fasteners to which an electronic device can be attached for receiving data from said one or more sensors, a first textile layer which in use is on the inside of the garment, a second textile layer which in use is on the outside of the garment, and a third intermediate layer provided between said first and second layer along at least a portion of said tube, wherein said first and second textile layers are made from a substantially elastic material, and said third intermediate layer is substantially more rigid than said first and second textile layers. The sensors may be ECG and respiration sensors.

The present invention relates to a sensor apparatus, and more particularly relates to a sensor apparatus which is adapted to be integrated in a garment.

It is known that athletes want to monitor their own physical status when doing exercise, whether this exercise is in the form of competition or during training. Many runners for example carry with them a Velcro breast strap for monitoring their heart rate. Others use a heart rate monitor on their wrist. Although these systems do allow an athlete to gather some information during exercise or after their exercise, they suffer from several disadvantages: firstly, the heart rate monitors can easily be forgotten and secondly, they are not always very comfortable in their use. Thirdly, only very limited information, namely only the heart rate, is available.

To resolve these issues, garments have been provided with integrated sensors. EP 1 916 323 discloses a garment which comprises a band portion having electrodes in contact with the skin of a user. An electronic device can be incorporated in the garment to measure and process signals from the electrodes in contact with the skin.

WO 02/071935 discloses a garment (such as a bra or undershirt) with a sensor positioned in the chest area of the garment. Press studs are provided, to which a transmitter can be connected.

US 2005/0010096 discloses a garment to which a plurality of ECG pickup contacts are attached. The garment furthermore comprises snaps for connection with ECG sensors.

These prior art garments resolve some of the aforementioned problems. A sensor integrated in a garment cannot be forgotten and in general may be more comfortable in use. However, there still exists a need for a garment that may comprise a plurality of sensors, which is comfortable to use and to which an electronic device may easily be connected. There also still exists a need for such a garment which is reliable in use, is relatively cheap and is user-friendly (in that it can e.g. easily be washed in a washing machine). There further exists a need for a garment which can be used in combination with e.g. a mobile phone and/or a web portal to further help athletes before, during and after exercise.

It is an object of the present invention to at least partially fulfil these needs. The present invention is particularly aimed at runners, joggers, racewalkers, but may be used by anyone involved in any sort of physical exercise. Furthermore, the present invention may also be advantageously used by e.g. the chronically ill, people or patients known to suffer from cardiac problems, and patients recovering from a surgical intervention or cardiac episode or a disease. Furthermore, the invention may be used in the industrial sector as a security product for people or operators with a high level of responsibility or risk such as fire fighters, harbour operators, refinery operators, police, etc.

In a first aspect, the present invention provides a sensor apparatus adapted to be incorporated in a garment, said apparatus comprising one or more sensors, one or more fasteners to which an electronic device can be attached for receiving data from said one or more sensors, a first textile layer which in use is on the inside of the garment, a second textile layer which in use is on the outside of the garment, and a third intermediate layer provided between said first and second layer along at least a portion of said tube, wherein said first and second textile layers are made from a substantially elastic material, and said third intermediate layer is substantially more rigid than said first and second textile layers.

In this aspect, the elastic layers may be more comfortable to wear for users and provide the flexibility which is needed for the proper functioning of various sensors. The intermediate more rigid layer provides the appropriate stiffness so that an electronic device which is aimed at receiving data from the sensors can more easily be attached to the apparatus (and garment). Suitable materials for the intermediate layer may include e.g. Teflon™-based textiles (such as Gore-tex™), silicon heat transfer substrates or plastic heat transfer substrates, or relatively rigid textiles.

In the context of the present invention, a textile is to be understood to comprise any kind of woven, knitted, or tufted cloth, or a non-woven fabric (e.g. a cloth made of fibres that have been bonded into a fabric). Textile in this context furthermore also comprises yarns, threads and wools that can be spun, woven, tufted, tied and otherwise used to manufacture cloth. Also in the present context, “elastic material” is to be understood as a material which relatively easily may be stretched or compressed and is able to resume its original shape.

The combination of two elastic textile layers on top of each other establishes that the outer elastic layer exerts some force on the inner elastic layer, so that the inner layer is pressed against a user's skin. In embodiments wherein one or more sensors are provided in a first textile layer, these sensors may thus be pressed into good contact with a user's skin.

In some embodiments of the invention, the elastic textile layers comprise e.g. lycra, and or polyamide, and/or nylon and/or Thermocool™. In other embodiment, other textiles may be used.

In some embodiments, said third intermediate layer is only provided in a portion of the apparatus comprising said one or more fasteners. The more rigid third intermediate layer mainly serves to provide a zone in which an electronic device may easily be mounted. The use of materials may be minimized by limiting the extent of the third textile layer to this zone.

In some embodiments, said third intermediate layer is substantially impermeable to water. In these embodiments, potential short circuits caused by excessive transpiration of a user may be avoided.

In some embodiments, the sensor apparatus comprises at least two ECG sensors. In preferred embodiments, said at least two ECG sensors are provided in said first textile layer, substantially in direct contact with a user's skin. Optionally, said at least two ECG sensors are made from conductive yarn. Suitable conductive yarns may comprise e.g. cotton, polyamide, polyester, or nylon substrates embedded with electrically conductive elements, like e.g. carbon, nickel, copper, stainless steel, gold, silver, or titanium. In other embodiments, other suitable skin electrodes could be used. In some embodiments, the ECG electrodes may also be covered with a silicon patch.

In some embodiments, one or more of said fasteners are positioned substantially between the two ECG sensors. In some embodiments, it may be beneficial to position the ECG sensors with a substantial distance between them such that in use a suitable difference in potential is established.

In some embodiments, said at least two ECG sensors are connected to one or more of said fasteners through conductive yarn. In other embodiments of the invention, e.g. electrical wiring may be used to connect the ECG sensors to one or more of the fasteners.

In some embodiments, the sensor apparatus further comprises a respiration sensor. In other embodiments, other sensors may (also) be used such as e.g. a thermometer, skin resistance sensor, transpiration sensor or other. In some embodiments, said respiration sensor is provided in said second textile layer. By providing said respiration sensor in an elastic layer, it may deform more easily and the respiration sensor may be adapted to determine a user's respiration by registering the sensor's variation in electrical resistance due to its deformation in use.

In some embodiments, said respiration sensor is provided with at least one of said fasteners. Preferably, a fastener is provided at each end of the respiration sensor. In some embodiments, said respiration sensor substantially has a shape of an open rectangle. It has been found that in combination with two ECG sensors, an advantageous arrangement of the electronic device can be provided using a respiration sensor which substantially has a shape of an open rectangle.

In some embodiments, said fasteners are press studs (also sometimes referred to as snaps, snap fasteners, or poppers). Such press studs are commonly made of a pair of interlocking discs. A circular lip under one disc fits into a groove on the top of the other, holding them fast until a certain amount of force is applied. The press studs may be attached to fabric by hammering, plying, or sewing. In other embodiments, other kinds of fasteners may be used, such as e.g. magnets, a pin-socket or plug-socket connection (e.g. with the socket being provided on the sensor apparatus), conductive Velcro, or other conductive metal clip fasteners. Preferably, any kind of fastener that allows an electronic device to be easily attached and detached may be used.

In some embodiments, in use said electronic device is attached on the outside of the garment. In these embodiments, the electronic device may be particularly easily attached and detached by a user, even possibly during exercise.

In another aspect, the invention provides a garment comprising a sensor apparatus substantially as hereinbefore described. In preferred embodiments, said sensor apparatus is arranged in said garment such that in use the apparatus is arranged substantially in the area immediately below a user's chest, or in the area of intersection between the chest and abdomen. These areas may be suitable locations for the measuring of various parameters, notably a user's respiration and ECG. Depending however on the sensors used and the exact positions of the sensors in the sensor apparatus, a slightly different area (or a larger area) may be more suitable.

In some embodiments, the other parts of said garment are made with a single textile layer. In embodiments of the invention, said garment may comprise a single textile layer, which forms either the first or the second textile layer of the sensor apparatus substantially as hereinbefore described. In these embodiments, e.g. the sleeves or straps, or collar or the garment may comprise more than one layer.

Within the scope of the invention, the sensor apparatus may be incorporated in any garment. In preferred embodiments of the invention, the sensor apparatus may be integrated in e.g. a t-shirt with long sleeves, a t-shirt with short sleeves, a tank top, a leotard, a bra, a sleeveless top, an A-shirt, a halter top, a spaghetti-strapped shirt, a singlet, or a tube top.

In yet another aspect, the invention provides a method of manufacturing a sensor apparatus, comprising providing a first and a second textile layer, said first and second layers being made from a substantially elastic material, and at least one of said first and second textile layers comprising at least one sensor; providing a third intermediate layer between said first and second textile layers, said third intermediate layer being substantially more rigid than said first and second textile layers; heat pressing said third intermediate layer to said second textile layer; mounting one or more fasteners to which an electronic device can be attached on said second and/or third layer and; sewing said first and second textile layers together.

In this context, sewing is to be understood to comprise any form of joining or attaching textiles using yarn or thread. Particularly, it is meant to include stitching, weaving, knitting or embroidering of any kind.

In yet a further aspect, the invention provides a method of manufacturing a main portion of a garment comprising providing a base textile layer, said base textile layer being made from a substantially elastic material, said base textile layer substantially having a shape of said main portion of said garment; providing an additional textile layer along a portion of said base textile layer, said additional textile layer being made from a substantially elastic material and at least one of said base and additional textile layers comprising at least one sensor; providing an intermediate layer between said base and additional textile layers, said intermediate layer being substantially more rigid than said base and additional textile layers; heat pressing said intermediate layer to said base and/or additional textile layer; mounting one or more fasteners to which an electronic device can be attached to said base and/or additional and/or intermediate layer; and sewing said base and additional textile layers together. In this sense, the base and additional textile layer may be either respectively the first and second textile layers or vice versa as previously hereinbefore described with reference to the sensor apparatus.

Particular embodiments of the present invention will be described in the following, only by way of non-limiting examples, with reference to the appended drawings, in which:

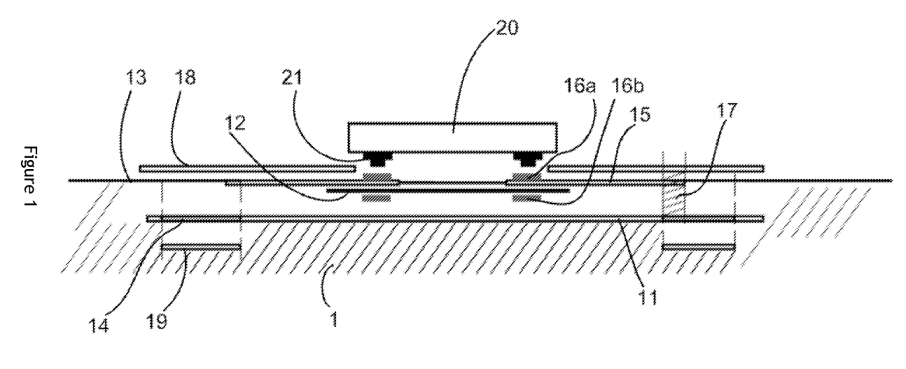

FIG. 1 illustrates a cross-section of an embodiment of a sensor apparatus according to the present invention;

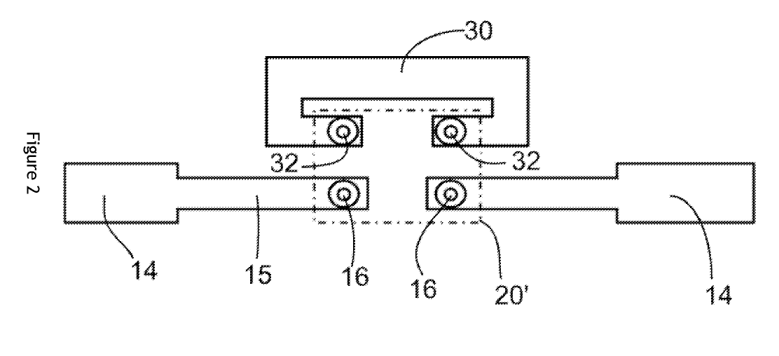

FIG. 2 schematically illustrates a frontal view of an embodiment of a sensor apparatus according to the present invention;

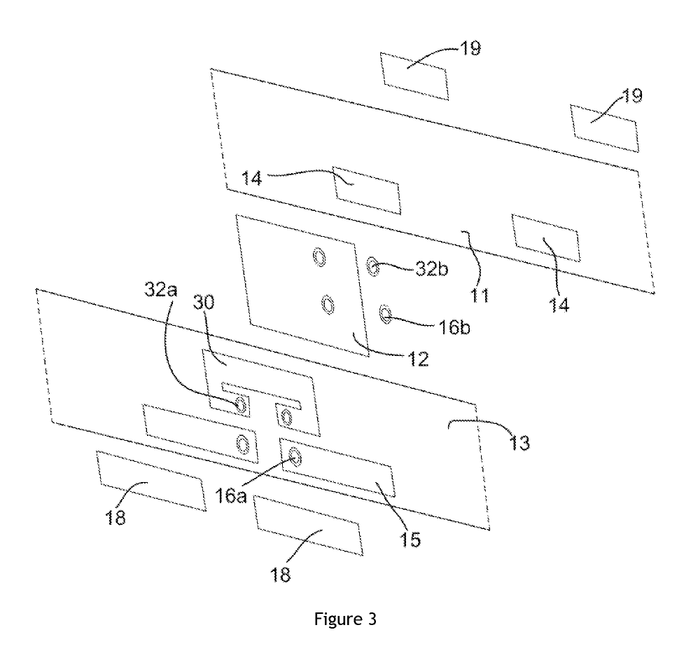

FIG. 3 schematically illustrates the built-up of a sensor apparatus according to an embodiment of the present invention;

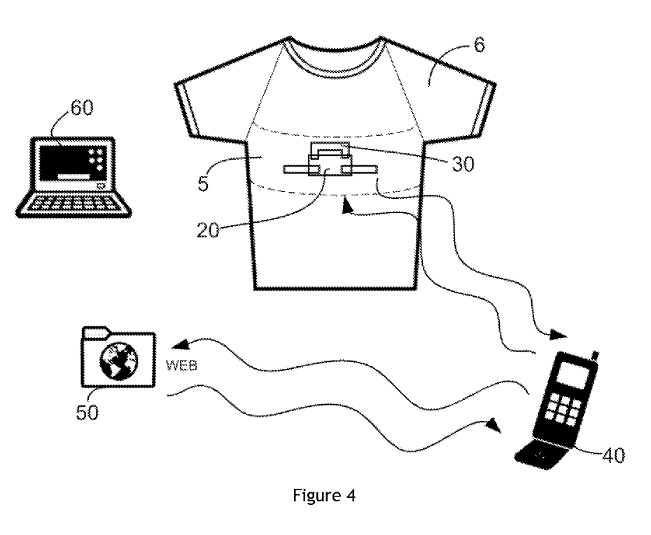

FIG. 4 schematically illustrates a garment according to an embodiment of the present invention and its use in combination with an electronic device, mobile phone and web page.

FIG. 1 illustrates a cross-section of an embodiment of a sensor apparatus according to the present invention. It will be clear from the explanation that follows that the drawings are schematic, and that the distances, and thicknesses of elements are in some cases exaggerated or otherwise misrepresented. The chosen thicknesses and distances in the figure have merely been selected to more clearly illustrate the various aspects of the invention.

A sensor apparatus (in this embodiment shaped like a textile tube) comprises a first textile layer 11, which in use is in contact with the user's skin 1. The apparatus further comprises a second textile layer 13 (which in use is on the outside of a garment) and an intermediate third layer 12.

Said first layer 11 and second layer 13 are made of a substantially elastic material. In preferred embodiments, said first layer 11 and second layer 13 may be made from permeable (“breathing”) materials. As an example of a suitable elastic material, a combination of Thermocool™ (commercially available from e.g. Advansa™), nylon and lycra may be used. In a particular example, the elastic material consists of 70% Thermocool™, 20% nylon and 10% lycra. In other embodiments of the invention however, these same materials in other proportions may be used. In other preferred embodiments, yet completely different materials may be used. One way of manufacturing the elastic textiles is by seamless sewing. During the manufacture of elastic textiles, the tension of the yarn used may be manipulated. This way certain parts of a garment may fit more tightly around a user's body than other parts. For example, it is not desirable that a garment is very tight in the area of an armpit of a user, whereas a fit in a chest portion can be tighter.

In the embodiment of FIG. 1, two ECG sensors 14 are provided in first textile layer 11. These ECG sensors may be made of conductive yarns which may be sewn or stitched onto said textile layer 11. Further in this particular embodiment, silicon patches 19 may be provided. These silicon patches may serve to ensure that the ECG sensors do not move or slide too much with respect to the user's body. In other embodiments of the invention, the apparatus does not comprise such silicon patches.

Further in the embodiment of FIG. 1, electrical paths or “tracks” 15 are provided in the second textile layer 13 to electrically connect the ECG sensors 14 to fasteners 16. In this embodiment, the fasteners are press studs. The female parts of the press studs 16 comprise top part 16a provided in second textile layer 13, and bottom part 16b provided below intermediate textile layer 12. The male parts 21 of the press studs are provided on an electronic device 20 which may be attached to the sensor apparatus and garment. Suitable materials for the tracks 15 include include e.g. cotton, polyamide, polyester, or nylon substrates embedded with electrically conductive elements, like e.g. carbon, nickel, copper, gold, silver, stainless steel or titanium. In one embodiment, conductive yarn of 70% polyamide and 30% stainless steel may be used (the polyamide being provided around the stainless steel wires). In another embodiment conductive yarn of 50% polyamide and 50% stainless steel may be used. In yet further embodiments, a 100% stainless steel yarn or wire could be used for the ECG sensors and/or tracks. Reference sign 17 has been used to indicate the connection by stitching or sewing conductive yarn between the ECG sensors 14 to tracks 15. Although not shown in FIG. 1, such stitching or sewing is provided for both ECG sensors 14 of FIG. 1.

The electronic device 20 may be used for receiving and collecting and/or storing and/or processing, and/or transmitting data from the sensors. The electronic device 20 may further incorporate other sensors such as an accelerometer, preferably a three-dimensional accelerometer. Data from the ECG sensors, respiration sensor, accelerometer and optionally other sensors may be transmitted and/or analysed to assist in the training of a user.

In the embodiment of FIG. 1, press studs were used as fasteners. It will be clear that any other suitable fastener may also be used, as long as an electrical connection can be established between the sensors and the fasteners. An example of an alternative fastener may e.g. be conductive Velcro.

In the embodiment of FIG. 1, the sensor apparatus comprises two ECG sensors. Within the scope of the invention however, the sensor apparatus may comprise other sensors, such as e.g. thermometer, skin resistance sensor, transpiration sensor, respiration sensor, a plurality of these, or yet other sensors.

During the manufacture of the sensor apparatus, the intermediate layer may preferably be heat pressed between the first and second textile layers.

The intermediate layer 12 may be substantially more rigid than the first and second textile layers 11 and 13. The intermediate layer may thus locally provide a rigid zone, to which the electronic device may be easily attached. By only providing the intermediate layer along a portion of the sensor apparatus, the other parts of the apparatus may be more elastic or flexible, which may be more comfortable for users and also can enhance the performance of the respiration sensor (not shown in FIG. 1). Suitable materials for the intermediate layer include e.g. Teflon™. In other embodiments, silicon heat transfer substrates or plastic heat transfer substrates, rubber, or relatively rigid textiles may be used. It should be understood that although it is desirable that the intermediate layer is substantially more rigid than the first and second textile layers, such rigidity will also have limits in order to safeguard a user's comfort.

Preferably, the intermediate layer 12 is impermeable to water. This may avoid e.g. short circuits caused by excessive transpiring by a user.

Finally, in this embodiment, a heat press transfer substrate 18 may be heat pressed onto the outer layer 13. Such substrate may be decorative and e.g. comprise a logo, a drawing, a trademark name or may simply be used to mask the tracks 15. In other embodiments of the invention, such a heat press transfer substrate is not necessarily provided.

The sensor apparatus may be formed substantially as a tube, substantially completely surrounding a part of a user's torso. In other embodiments, the sensor apparatus may only comprise a portion of a tube and may e.g. be formed more like a strip or band.

FIG. 2 schematically illustrates a frontal view of an embodiment of a sensor apparatus according to the present invention. In this embodiment, a respiration sensor 30 has been provided, in combination with two ECG sensors 14. This respiration sensor may have a shape resembling a rectangle, which is open on one of its longer sides. At its ends, fasteners 32 (in the shown embodiment, female parts of press studs) may be provided.

In this embodiment, the respiration sensor is provided above the ECG sensors 14, and the “tracks” 15 connecting the ECG sensors to fasteners 16. By suitable positioning of the open rectangle with respect to the ECG sensors and tracks, a rectangular area 20′ is created of suitable dimensions for an electronic device.

In preferred embodiments of the invention, the electronic device which may be attached to the sensor apparatus (and garment) will be substantially small and light weight so that it is comfortable for a user to carry on the garment. However, the ECG sensors 14 are preferably placed a suitable distance apart to create a notable difference in potential between the ECG sensors, such that their signals can be distinguished. Additionally, respiration sensor 30 has a suitable length such that movement of the user's thorax can be detected by (directly or indirectly) measuring the deformation of the respiration sensor.

In some embodiments of the invention, the respiration sensor comprises piezoresistive carbon covered fibres. Its deformation can be detected by measuring its electrical resistance. A change in its electrical resistance indicates a change in length of the sensors and thus indicates expansions or contraction of the thorax. It is thus advantageous to provide the respiration sensor in an elastic textile layer. Such an elastic textile layer may deform relatively easily with the movements of a user's thorax. The electronic device which is to be connected to the sensors may require some rigidity for its mounting. A third intermediate layer of suitable material may provide such rigidity locally and not impair the proper functioning of e.g. the respiration sensor.

With the shape of the respiration sensor, the use of the tracks 15, and the positioning of FIG. 2, the ECG sensors and respiration sensor may function properly, while the electronic device may be of reduced dimensions and can still be easily attached to the garment. In preferred embodiments, the respiration sensor is thus positioned substantially between the ECG sensors. In this sense, the respiration sensor does not need to be positioned on an imaginary straight line between said ECG sensors, but may advantageously be provided above (or in some alternative embodiments, below) such an imaginary line.

FIG. 3 schematically illustrates the built-up of a sensor apparatus according to an embodiment of the present invention and may also be used to schematically illustrate a method of manufacturing a sensor apparatus according to the present invention. First textile layer 11 comprises the ECG sensors 14, preferably made of conductive yarn. Optional silicon patches 19 may be positioned between the skin of the user and the ECG sensor.

In this embodiment, bottom parts of the female parts of the press studs, 32b and 16b are provided underneath intermediate layer 12. Finally, second layer 13 is shown with respiration sensor 30 and the “tracks” (“electrical paths”) 15. Top parts of the female parts of the press studs 16a and 32a are provided on top of second layer 13.

In a preferred method of manufacturing a sensor apparatus, said third intermediate layer is heat pressed and connected to said second textile layer. Subsequently, said first and second textile layers may be sewn together. Preferably, before said intermediate layer is heat pressed between said first and second layer, ECG sensors 14 may be connected to tracks 15, by stitching or sewing with conductive yarn.

In accordance with the invention, a main portion of a garment may be manufactured by providing a first base textile layer 11, said first textile layer being made from a substantially elastic material, said base textile layer 11 having the shape of the main portion of the garment. Subsequently a second textile layer may be provided along a portion of said first textile layer, said second textile layer being made from the same of different elastic material, and a third intermediate layer may be provided between said first and second textile layers, said third intermediate layer being substantially more rigid than said first and second textile layers. Then, third intermediate layer may be heat pressed to said second textile layer. Then, one or more fasteners (such as e.g. press studs, sockets (of e.g. a jack connector), clips, conductive Velcro etc.) may be mounted on said second and/or third layer. Then, said first and second textile layers may be sewn together. To finish the manufacturing of the garment, other parts of the garment such as e.g. sleeves, straps, or collar may be sewn to the main portion.

Finally, also in FIG. 3, optional decorative heat transfer substrates 18 are shown.

In FIG. 3, first layer 11 and second layer 13 have been indicated as strips without ends. In some implementations, both layers may form complete tubes. In other implementations, one or both of the layers may not form a complete tube, and rather be formed like e.g. rectangular strips.

In the embodiments of FIGS. 1-3, the sensor apparatus was provided with four press studs. It will be clear however, that a different number of press studs may also be used. This number of press studs (or other type of fasteners) may e.g. vary with the number and kind of sensors incorporated in the sensor apparatus.

FIG. 4 schematically illustrates a garment 6 according to an embodiment of the present invention. The garment comprises a tubular sensor apparatus 5 substantially as hereinbefore described. The sensor apparatus 5 may comprise a respiration sensor 30, ECG sensors (not visible, not shown) and an electronic device 20. The respiration sensor has a slightly different shape than shown in e.g. FIG. 3. The respiration sensor 30 in this embodiment has an inverted U-shape (or rotated C-shape).

FIG. 4 also illustrates a garment 6 with integrated sensors and its use in combination with an electronic device 20, mobile phone 40 and web page 50. The electronic deice is adapted to receive data from sensors incorporated in garment 6. For example, an ECG signal may be received. The electronic device may further be adapted to process said signal, and provide an improved ECG signal after e.g. filtering out noise.

The electronic device may further comprise storage means, such as e.g. a MicroSD memory card for storing data from the sensors obtained before, and/or during and/or after exercising. It may also conveniently comprise one or more batteries.

The electronic device may further comprise a Bluetooth module for communicating with a mobile phone 40. Data collected from the sensors may thus be transmitted to a user's mobile phone. As was mentioned before, also the electronic device may comprise further sensors such as e.g. a three-dimensional accelerometer or a GPS system. Data from this accelerometer, GPS and/or other sensors may also be transmitted to a user's mobile phone. Electronic device 20 may further comprise means for connecting it to a computer (such as a PC, Mac, laptop or similar). For this purpose, it may e.g. comprise a micro USB port, a normal USB port, a FireWire port or similar. Such a connection with a computer may be used to transfer data from the computer to the electronic device or vice versa or for charging the battery or batteries of the electronic device.

It will be clear that a garment with incorporated sensors, in combination with an electronic device, and optionally in combination with a mobile phone (or similar device), may serve a useful purpose for athletes. Before, during and after physical exercise, they can monitor e.g. their ECG, respiration rate, heart rhythm, speed, distance covered during exercise, calories burned and R-R interval (period of respiration). However, this kind of information may also be useful for e.g. the chronically ill, people or patients known to suffer from cardiac problems, and patients recovering from a surgical intervention or cardiac episode or a disease. The sensor apparatus according to the invention and a garment incorporating such a sensor apparatus may even be useful in an industrial environment, wherein it is important to monitor the physical condition of employees.

Mobile phone 40 may further be provided with a computer program (software) that may be adapted to e.g. collect data from the electronic device, and/or process said data to monitor a user's performance before, during or after exercise, and/or compare said data to a preloaded exercise program, and/or transmit data related to a user's exercise to a dedicated web page. Processing the data from sensors may e.g. comprise filtering out noise from the sensors which was not filtered by the electronic device. The mobile phone may comprise a Bluetooth module to be able to communicate with the electronic device.

The mobile phone may also be adapted to be able to control the electronic device. If the mobile phone is provided with GPS functionalities, data from the GPS module may be combined with data from the sensors in the garment and/or sensors in the electronic device. The mobile phone may furthermore be adapted to present data related to exercises in a suitable form e.g. with graphics relating to pulse rate, ECG, average speed, total distance covered etc.

Although hereinbefore reference was made in general to a mobile phone, it will be clear that a mobile phone may be substituted by a suitable PDA, iPod™ iPhone™, Blackberry™, Smartphone or other similar device (as long as functionalities are incorporated which allow communication with electronic device 20).

It is furthermore preferred that the mobile phone is adapted to upload and/or download data from a dedicated website 50.

Said website 50 may be adapted to allow users to login and collect data related to their exercises. The website may furthermore allow user to download e.g. running schedules, suitable routes for running and an exercise to be performed. The suggested schedules, exercises and routes may e.g. be based on a user's previous exercises, and data that was collected from the sensors during these previous exercises. The suggested schedules and/or routes may furthermore be based on other personal data a user can provide at the website (e.g. the desire to loose weight, to run a half marathon, a complete marathon or to work on endurance). The suggested schedules and/or may also be based on other personal data such as e.g. sex, age, weight, height, cardiac fitness, smoker or non-smoker.

In some embodiments of the invention, the electronic device 20 is adapted to collect data from the sensors, and transmit them to a mobile phone (or similar device). The mobile phone may comprise a computer program that is adapted to compare the data received from the electronic device with a preloaded exercise schedule. Based on this comparison, the mobile phone may give suggestions or instructions to a user. For example, on the display of the mobile phone, an instruction such as “pause”, “accelerate”, “decelerate” may be shown. Alternative such an instruction could be communicated to a user through headphones connected to the mobile phone.

It will be clear that a dedicated website may further be completed with the ability to e.g. upload photos, connect with other users of similar profile, suitable graphic representation of various parameters collected before, during or after exercise.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described before, but should be determined only by a fair reading of the claims that follow.

1. A sensor apparatus adapted to be incorporated in a garment,
said apparatus comprising one or more sensors, one or more fasteners to which an electronic device can be attached for receiving data from said one or more sensors, a first textile layer which in use is on the inside of the garment, a second textile layer which in use is on the outside of the garment, and
a third intermediate layer provided between said first and second layer along at least a portion of said apparatus,
wherein said first and second textile layers are made from a substantially elastic material, and
said third intermediate layer is substantially more rigid than said first and second textile layers.

2. A sensor apparatus according to claim 1, wherein said third intermediate layer is only provided in a portion of the apparatus comprising said one or more fasteners.

3. A sensor apparatus according to claim 1, wherein said third intermediate layer is substantially impermeable to water.

4. A sensor apparatus according to claim 1, further comprising at least two ECG sensors in said first textile layer.

5. (canceled)

6. A sensor apparatus according to claim 4, wherein said at least two ECG sensors are made from conductive yarn.

7. A sensor apparatus according to claim 4, wherein one or more of said fasteners are positioned substantially between the two ECG sensors and wherein the ECG sensors are connected to one or more of the fasteners through conductive yarn.

8. (canceled)

9. A sensor apparatus according to claim 1, further comprising a respiration sensor in said second textile layer.

10. (canceled)

11. A sensor apparatus according to claim 9, wherein said respiration sensor is adapted to determine a user's respiration by registering the sensor's variation in electrical resistance due to its deformation in use.

12. A sensor apparatus according to claim 9, wherein said respiration sensor substantially has a shape of an open rectangle.

13. A sensor apparatus according to claim 9, wherein said respiration sensor is provided with at least one of said fasteners and wherein one fastener is provided at each end of the respiration sensor.

14. (canceled)

15. A sensor apparatus according to claim 1, wherein said fasteners are press studs and wherein in use said electronic device is attached on the outside of the garment.

16. (canceled)

17. A sensor apparatus according to claim 1, wherein said apparatus is substantially tubular.

18. A garment comprising a sensor apparatus according to claim 1.

19. A garment according to claim 18, wherein said sensor apparatus is arranged in said garment such that in use the apparatus is arranged substantially in the area immediately below a user's chest.

20. A garment according to claim 18, wherein other parts of said garment are made with a single textile layer.

21. (canceled)

22. A method of manufacturing a sensor apparatus, comprising
(a) providing a first and a second textile layer, said first and second layers being made from a substantially elastic material and at least one of said first and second textile layers comprising at least one sensor,
(b) providing a third intermediate layer between said first and second layers, said third intermediate layer being substantially more rigid than said first and second textile layers,
(c) heat pressing said third intermediate layer to said second textile layer, and
(d) mounting one or more fasteners to which an electronic device can be attached on said second and/or third layer, and
(e) sewing said first and second textile layers together.

23. A method of manufacturing a sensor apparatus according to claim 22, wherein at least one ECG sensor is provided in said first textile layer and further comprising sewing at least one conductive yarn to electrically connect at least one ECG sensor to at least one fastener.

24. (canceled)

25. (canceled)

26. A method of manufacturing a sensor apparatus according to claim 23, wherein said conductive yarn to electrically connect at least one ECG sensor to at least one fastener is sewn in said second textile layer.

27. A method of manufacturing according to claim 22, wherein said fasteners are female press studs, one half of said female press studs being mounted below said third intermediate layer, and the other half of said female press studs being mounted on top of said second textile layer.

28. A method of manufacturing a main portion of a garment comprising
(a) providing a base textile layer, said base textile layer being made from a substantially elastic material, said base textile layer substantially having a shape of said main portion of said garment,
(b) providing an additional textile layer along a portion of said base textile layer, said additional textile layer being made from a substantially elastic material and at least one of said base and additional textile layers comprising at least one sensor,
(c) providing an intermediate layer between said base and additional textile layers, said intermediate layer being substantially more rigid than said base and additional textile layers,
(d) heat pressing said intermediate layer to said base and/or additional textile layer, and
(e) mounting one or more fasteners to which an electronic device can be attached to said base and/or additional and/or intermediate layer, and
(f) sewing said base and additional textile layers together.
